(19) United States
(12) Patent Application Publication
(10) Pub. No.: US 2024/0241126 A1
(43) Pub. Date: Jul. 18, 2024

(54) BIOCHIP AND METHOD FOR TRACKING POSTOPERATIVE RECURRENCE STATUS OF PATIENT WITH LUNG ADENOCARCINOMA

(71) Applicants: National Taiwan University, Taipei (TW); Phoenix Silicon International Co., Hsinchu City (TW); MacKay Medical College, New Taipei City (TW)

(72) Inventors: Szu-Hua Pan, New Taipei City (TW); Yuan-Ling Hsu, New Taipei City (TW); Ching-Wen Li, Hsinchu City (TW); How-Wen Ko, Taoyuan City (TW); Chung-Lieh Hung, New Taipei City (TW)

(21) Appl. No.: 18/352,651

(22) Filed: Jul. 14, 2023

(30) Foreign Application Priority Data
Jan. 18, 2023 (TW) 112102153

(51) Int. Cl. G01N 33/574

(52) U.S. Cl. CPC G01N 33/57423; G01N 2800/52; G01N 2800/7028

(57) ABSTRACT

The present invention provides a biochip for tracking postoperative recurrence status of a patient with lung adenocarcinoma. The biochip comprises a bare plate layer, the bare plate layer comprises a sensing electrode, and the sensing electrode comprises a biological agent capable of measuring an expression amount of a GPNMB gene. The present invention further provides a method for tracking the postoperative recurrence status of a patient with lung adenocarcinoma. The method comprises the following steps: step one, providing a sample from a patient with lung adenocarcinoma; step two: contacting the sample with a carrier capable of detecting an expression amount of a GPNMB gene; and step three: analyzing a change of the expression amount of the GPNMB gene to track the postoperative recurrence status of the patient with lung adenocarcinoma. The present invention utilizes a mass production capability of a semiconductor lithography process to control a chip cost, achieves an effect of a quick examination by using one drop of blood, can track the postoperative recurrence status of a patient with lung adenocarcinoma in real time so as to perform early treatment, and reduces a medical cost of the patient with lung adenocarcinoma.

### REFERENCE TO RELATED APPLICATIONS

The present application is based on, and claims priority from, Taiwan application number 112102153, filed Jan. 18, 2023, the disclosure of which is hereby incorporated by reference herein in its entirety.

### BACKGROUND OF THE INVENTION

### Field of the Invention

The present invention relates to a biochip and a method for tracking postoperative recurrence status of a patient with lung adenocarcinoma, particularly a biochip for tracking a recurrence due to a drug resistance produced in a patient with lung adenocarcinoma after a target drug therapy.

### Description of the Prior Art

A current diagnosis of a patient with lung adenocarcinoma or tracking of a recurrence of a patient is mainly based on an X-ray imaging diagnosis. A blood diagnosis is performed using a next generation nucleotide sequencing (NGS) or an enzyme-linked immunosorbent assay (ELISA) as detection methods. The former is to analyze an epithelial growth factor receptor (EGFR) mutation in DNA for a determination and each analysis costs one hundred thousand New Taiwan dollars. The latter is to analyze changes of protein expression amounts of biomarkers (CEA, Cyfra21-1, NSE, etc.) in blood. An ELISA set for a single biomarker is about twenty thousand New Taiwan dollars. Both detection and analysis require one day. Besides, it takes patients 3 to 7 days to obtain detection results. In summary, it is time-consuming (requiring frequently roundtrip to a hospital) and costly if a traditional method is used to track the postoperative recurrence status of patients. In addition, since patients need to frequently roundtrip to the hospital, the hospital is usually overcrowding. From a prevention perspective of an infectious disease (ex: novel coronavirus COVID-19), a risk of spreading the infectious disease is certainly increased. Therefore, it is necessary to develop a detection chip which can be rapid, real-time and affordable during a diagnosis.

Glycoprotein nonmetastatic melanoma protein B (GPNMB) is a type I transmembrane protein. The gene is positioned on a seventh chromosome of a human body and the protein consists of 572 amino acids. An intact transmembrane protein can be cleaved by an extracellular protease such as ADAM10 to release an extracellular segment of GPNMB into blood to play a role as a cytokine. GPNMB can be detected in normal bones, hematopoietic system, skin tissues, etc., and can also be detected in malignant hyperplasia tissues such as breast cancer, melanoma and hepatocellular carcinoma at a high level. However, no reference exists to date that indicates a correlation between an expression amount of a GPNMB gene and lung adenocarcinoma.

In view of the above, the present inventor has well understood the deficiencies and defects of the prior art and is urgent to improve and innovate them. Besides, after several years of research, a biochip and a method for tracking the postoperative recurrence status of a patient with lung adenocarcinoma are successfully developed.

### SUMMARY OF THE INVENTION

In order to achieve the above objective, the present invention provides a biochip for tracking the postoperative recurrence status of a patient with lung adenocarcinoma, wherein the biochip comprises a bare plate layer, the bare plate layer comprises a sensing electrode, and the sensing electrode comprises a biological agent capable of measuring an expression amount of the GPNMB gene.

In one embodiment of the present invention, the biological agent measures an expression amount of an mRNA or a protein of the GPNMB gene.

In one embodiment of the present invention, the biological agent is an antibody specifically binding to a protein of the GPNMB gene.

In one embodiment of the present invention, the antibody has a concentration of 20 ng/ml-5 μg/ml.

In one embodiment of the present invention, the biochip for tracking the postoperative recurrence status of a patient with lung adenocarcinoma has a limit of detection of 0.5 ng/ml-70 ng/ml.

In one embodiment of the present invention, the biochip for tracking the postoperative recurrence status of a patient with lung adenocarcinoma further comprises a lower cover layer, a middle interlayer, and an upper cover layer, the lower cover layer is arranged at a lower side of the bare plate layer, and the middle interlayer and the upper cover layer are arranged on an upper side of the bare plate layer.

In one embodiment of the present invention, the bare plate layer comprises a plurality of electric wires, a relative electrode, and a reference electrode, the middle interlayer comprises a micro-channel, and the upper cover layer comprises a sample dripping area; and the micro-channel is arranged correspondingly to a position of the sample dripping area, such that a sample may flow from the sample dripping area to the sensing electrode.

In addition, the present invention provides a method for tracking the postoperative recurrence status of a patient with lung adenocarcinoma. The method comprises the following steps: step one, providing a sample from a patient with lung adenocarcinoma; step two: contacting the sample with a carrier capable of detecting an expression amount of the GPNMB gene to obtain expression amount information of the GPNMB gene; and step three: analyzing a change of the expression amount of the GPNMB gene to track the postoperative recurrence status of the patient with lung adenocarcinoma.

In one embodiment of the present invention, the sample is blood; and the expression amount of the GPNMB gene is a protein expression amount of the GPNMB gene.

In one embodiment of the present invention, the patient with lung adenocarcinoma has a mutation of L858R or L858R+T790M of EGFR; and the postoperative recurrence is due to development of a drug resistance of the patient with lung adenocarcinoma to a target drug, Iressa.

Using ELISA as a means for detecting a protein as a comparison target, the biochip for tracking a recurrence due to a drug resistance of a patient with lung adenocarcinoma after a target drug therapy is used for tracking a change of a postoperative recurrence of the patient with lung adenocarcinoma to perform an early treatment. In addition, a mass production capability of a semiconductor lithography process is utilized to control a chip cost, an effect of a real-time and quick examination by using one drop of blood is realized, and a medical cost of the patient with lung adenocarcinoma is reduced.

### BRIEF DESCRIPTION OF THE DRAWINGS

The techniques of present invention would be more understandable from the detailed description given herein below and the accompanying figures are provided for better illustration, and thus description and figures are not limitative for present invention, and wherein:

FIG. 1(A) is a schematic diagram of each layer of the biochip;

FIG. 1(B) is a schematic diagram of the assembled biochip not containing an upper cover layer;

FIG. 1(C) is a schematic diagram of the assembled biochip containing the upper cover layer;

FIG. 2(A) is a histogram of an expression amount of a GPNMB gene is highly correlated with a patient with lung adenocarcinoma in a pleural effusion sample;

FIG. 2(B) is a scattergram of an expression amount of a GPNMB gene is highly correlated with a patient with lung adenocarcinoma in a pleural effusion sample;

FIG. 3(A) is a histogram of the expression amount of the GPNMB gene is highly correlated with the patient with lung adenocarcinoma in a plasma sample;

FIG. 3(B) is a scattergram of the expression amount of the GPNMB gene is highly correlated with the patient with lung adenocarcinoma in a plasma sample;

FIG. 4 shows a modification change amount of a resistance value of each stage of a GPNMB protein captured by a GPNMB antibody modified on a surface of a sensing electrode;

FIG. 5(A) is resistance value changes of different expression amounts of the GPNMB protein on the biochip;

FIG. 5(B) is the expression amount of the GPNMB protein is linearly related to a resistance value;

FIG. 6(A) is a histogram of comparisons between the biochip according to one embodiment of the present invention and a conventional ELISA method;

FIG. 6(B) is a scattergram of comparisons between the biochip according to one embodiment of the present invention and a conventional ELISA method;

FIG. 7(A) is detection results of the biochip of one embodiment of the present invention for analyzing different GPNMB antibody concentrations in plasma samples of patients with lung adenocarcinoma, wherein an antibody concentration in FIG. 7(A) is 50 ng/ml;

FIG. 7(B) is detection results of the biochip of one embodiment of the present invention for analyzing different GPNMB antibody concentrations in plasma samples of patients with lung adenocarcinoma, wherein an antibody concentration in FIG. 7(B) is 200 ng/ml; and

FIG. 7(C) is detection results of the biochip of one embodiment of the present invention for analyzing different GPNMB antibody concentrations in plasma samples of patients with lung adenocarcinoma, wherein FIG. 7(C) is a control of the conventional ELISA method.

### DETAILED DESCRIPTION OF THE INVENTION

In the present description, many technical and scientific terms commonly used in the technical field of biochips are used extensively. In the following description, the following definitions are provided for clearly and consistently understanding the present description and the claims, as well as the scope of the terms to which they are entitled. Other terms not specifically defined below have meanings commonly understood by a person skilled in the art.

The terms “or”, “as well”, “and” used in the description, unless otherwise indicated, refer to “or/and”. Furthermore, the terms “including” and “comprising” are not intended to be limiting, but rather are to be construed in an open-ended manner. The preceding paragraph is merely a systematic reference and should not be construed as limiting the subject matter of the present invention.

“%” used in the description refers to “weight percentage (wt %)” unless otherwise specified; a numerical range (e.g., 10%-11% of A) includes upper and lower limits (i.e., 10%≤A≤11%) unless otherwise specified; a numerical range without defining a lower limit (e.g., less than 0.2% of B or below 0.2% of B) means that the lower limit may be 0 (i.e., 0%≤B≤0.2%); and a ratio relationship of “a weight percentage” of each component may be replaced by a ratio relationship of “parts by weight”.

All numerical numbers disclosed in the present description may have a standard technical error of measurement (standard deviation) of ±10%. The term “about” is intended to indicate ±10%, ±5%, ±2.5%, or ±1% with respect to a given value, that is to say, “about” 20% represents 20±2%, 20±1%, 20±0.5%, or 20±0.25%.

The terms “treat”, “treating”, and “therapy” include alleviating, relieving or ameliorating at least one disease symptom or physiological status, preventing an additional symptom, inhibiting a disease or physiological status, arresting or slowing disease development, causing recovery of the disease or physiological status, and slowing the physiological status caused by the disease, and stopping the symptom or physiological status of the disease.

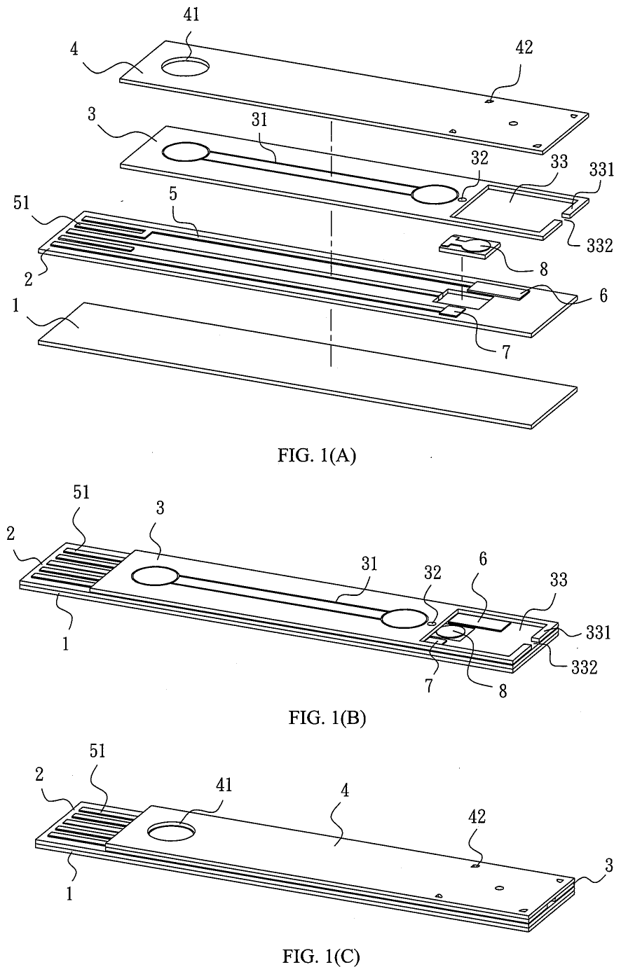

FIG. 1(A), FIG. 1(B) and FIG. 1(C) are a schematic diagram of a biochip according to one embodiment of the present invention. Referring to FIG. 1(A), FIG. 1(A) is the biochip of the present invention comprises a bare plate layer 2, the bare plate layer 2 comprises a sensing electrode 8, and the sensing electrode 8 comprises a biological agent capable of measuring an expression amount of the GPNMB gene.

In one embodiment, the bare plate layer 2 may include a chip groove 81 for arranging the sensing electrode 8. A material of the sensing electrode 8 is not limited and may be a silicon wafer, a metal, a glass, a plastic, etc. But from a viewpoint of improving stability of a GPNMB antibody immobilized on the biochip, it is preferable to use the silicon wafer or a Bare Au material. In addition, shapes, sizes, materials, etc., of the bare plate layer 2 and the chip groove 81 are well-known in the technical field of biochips, which will not be described in more detail herein.

In one embodiment, a biological agent capable of measuring an expression amount of the GPNMB gene is a biological agent capable of measuring an expression amount of an mRNA of the GPNMB gene, for example, may comprise a primer with fluorescence, a probe or any other biological agents well-known to be used for detecting the mRNA.

In one embodiment, the biological agent is an antibody specifically binding to a protein of the GPNMB gene, the sensing electrode 8 uses silicon as a substrate, the antibody is fixed on the substrate to form a biosensor, when an antigen (the GPNMB protein) in a sample is bound with the antibody, the biosensor transmits different electrical signals according to different contents of the GPNMB protein in the sample, and thus a protein concentration of the GPNMB gene in the sample is detected.

The antibody used in the biochip of the present invention may be any antibody that specifically binds to a protein of the GPNMB gene. A source, structure and sequence of the antibody are not limited, for example, a commercially available GPNMB antibody may be used. In one embodiment, the antibody of the present invention is a human bone activin/GPNMB antibody-Monoclonal Mouse IgG2B Clone #303822 (manufactured by bio-techne company, Cat. No. AF2550) or a human bone activin/GPNMB antibody-Antibody Affinity-purified Polyclonal Goat IgG (manufactured by bio-techne company, Cat. No. MAB25501).

The concentration of the antibody used in the biochip of the present invention is not limited and can be adjusted appropriately according to a structure of the biochip and a state of a patient with lung adenocarcinoma. In one embodiment, the concentration of the antibody is about 20 ng/ml-5 μg/ml, for example, may be about 30 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1 μg/ml, 2 μg/ml, 3 μg/ml, and 4 μg/ml, preferably about 50 ng/ml-2 g/ml, and more preferably about 50 ng/ml-200 ng/ml.

The expression amount of a human GPNMB gene detected by the biochip of the present invention may be any type of the GPNMB gene. A specific sequence of the GPNMB gene is not limited, for example, may be a gene of NCBI Gene ID: 10457. Similarly, the mRNA of the human GPNMB gene detected by the biochip of the present invention may be any type of the GPNMB mRNA, for example, may be mRNAs of NCBI Reference Sequence:NM_001005340.2, NM_002510.3, XM_005249578.4, XM_017011678.3, or XM_047419776.1. Furthermore, the protein of the human GPNMB gene detected by the biochip of the present invention may be any type of the GPNMB protein, for example, may be a protein of NCBI Reference Sequence: NP_001005340.1, NP_002501.1, XP_005249635.1, XP_016867167.1, or XP_047275732.1.

In one embodiment, the bare plate layer 2 comprises a plurality of electric wires 5, a relative electrode 6, and a reference electrode 7, one ends of a plurality of the electric wires 5 are connected with the relative electrode 6, the reference electrode 7, and the sensing electrode 8, the other ends of a plurality of the electric wires are connection ends 51, and the connection ends 51 are connected with a biochip detector (not shown in the figure); the material, length, etc., of the electric wires 5 are not limited, for example, the electric wires may be silver wires; and the material, length, etc., of the relative electrode 6 and the reference electrode 7 are not limited, for example, the relative electrode 6 may be a carbon electrode and the reference electrode 7 may be an Ag/AgCl electrode.

In one embodiment, the biochip of one embodiment of the present invention further comprises a lower cover layer 1, a middle interlayer 3, and an upper cover layer 4, the lower cover layer 1 is arranged at a lower side of the bare plate layer 2, and the middle interlayer 3 and the upper cover layer 4 are arranged on an upper side of the bare plate layer 2, wherein the middle interlayer 3 comprises a micro-channel 31 and the upper cover layer 4 comprises a sample dripping area 41; and the micro-channel 31 is arranged correspondingly to a position of the sample dripping area 41, such that a sample may flow from the sample dripping area 41 to the sensing electrode 8.

In one embodiment, the lower cover layer 1 is arranged at a lower side of the bare plate layer 2 to prevent the sensing electrode 8 of the bare plate layer 2 from contacting with the outside and maintain a mechanical strength of the biochip. The length and shape of the lower cover layer 1 may be the same as those of the bare plate layer 2.

In one embodiment, the middle interlayer 3 is arranged at an upper side of the bare plate layer 2 to provide a flow channel for the sample, for example, may provide a micro-channel 31 for guiding the sample to the sensing electrode 8 and prevent the sample from contacting a plurality of the electric wires 5 of the bare plate layer 2. The specific length, depth, shape, etc. of the micro-channel 31 are not limited. In another embodiment, the middle interlayer 3 may include a dispensing hole 32, for example, a dispensing hole 32 with a diameter of about 2 mm, and when dispensing is performed in the dispensing hole 32, an adhesive force between the middle interlayer 3 and the upper cover layer 4 may be increased, so as to improve the mechanical strength of the whole biochip.

In one embodiment, one end of the middle interlayer 3 may include a sensing area 33, for example, a sensing area 33 is arranged after a frame 331 of about 1 mm is reserved at one end of the middle interlayer 3, and the relative electrode 6, the reference electrode 7, and the sensing electrode 8 of the bare plate layer 2 on the lower side of the middle interlayer are exposed. In another embodiment, the sensing area 33 is not a sealed area, for example, a part of the frame 331 may be formed with a gap 332, such that the sensing area 33 may communicate with the outside air. By the above arrangement, the sample may be guided to the sensing area 33 smoothly for detection.

In one embodiment, the length of the middle interlayer 3 is shorter than that of the bare plate layer 2. When the middle interlayer 3 covers the bare plate layer 2, a part of the bare plate layer 2 is not covered by the middle interlayer 3, that is, one end of the bare plate layer 2 is exposed and called connection ends 51, and the connection ends 51 are used for connecting with the biochip sensor.

In one embodiment, the upper cover layer 4 is arranged at an upper side of the middle interlayer 3 to prevent the sample from contacting with the outside and maintain the mechanical strength of the biochip. The length and shape of the upper cover layer 4 may be the same as those of the middle interlayer 3. In one embodiment, the upper cover layer 4 may be provided with air holes 42 in positions corresponding to the sensing area 33 of the middle interlayer 3, for example, four air holes 42 with a diameter of about 1 mm may be arranged, such that the sensing area 33 may be more in circulation with the outside air. By the above arrangement, the sample may be guided to the sensing area 33 smoothly for detection.

In one embodiment, the upper cover layer 4 comprises a sample dripping area 41, the sample dripping area 41 is an inlet of the sample, and the sample enters from the sample dripping area 41 and passes through the micro-channel 31 to reach the sensing area 33. A form of the sample dripping area 41 is not limited and may be, for example, a hole, an injection port, a water-absorbent film, etc.

The shape, size, material, etc. of the lower cover layer 1, the middle interlayer 3, and the upper cover layer 4 are well-known in the technical field of biochips, which will not be described in more detail herein. FIG. 1(B) is a schematic diagram of the biochip according to one embodiment of the present invention (not containing the top cover layer 4). FIG. 1(C) is a schematic diagram of the biochip according to one embodiment of the present invention (containing the top cover layer 4).

After the sample enters from the sample dripping area 41 of the upper cover layer 4, the sample is guided to the sensing area 33 through the micro-channel 31 of the middle interlayer 3 and contacts with a biological agent on a sensing electrode 8. When a biological agent is an antibody, an antigen in the sample will react with the antibody. Besides, a relative electrode 6, a reference electrode 7, and a sensing electrode 8 transmits different electrical signals according to different contents of the GPNMB protein in the sample. The signals are transmitted to a biochip detector (not shown in the figure) through connection ends 51 by electric wires 5, and thus the content of the GPNMB protein in the sample is detected, a postoperative recurrence of a patient with lung adenocarcinoma can be tracked in real time to perform an early treatment, and a medical cost of the patient with lung adenocarcinoma is reduced.

In one embodiment, an LOD of the biochip of the present invention is not limited and can be adjusted appropriately according to a structure of the biochip, types and content of an antibody, etc. In one embodiment, the LOD of the biochip of the present invention is about 0.5 ng/ml-70 ng/ml, for example, may be about 1 ng/ml, 2 ng/ml, 5 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/ml, 60 ng/ml, and 65 ng/ml, preferably about 47.48 ng/ml.

The present invention further provides a method for tracking the postoperative recurrence status of a patient with lung adenocarcinoma, comprising the following steps:
step one, providing a sample from a patient with lung adenocarcinoma;
step two: contacting the sample with a carrier (for example, the biochip) capable of detecting an expression amount of a GPNMB gene to obtain expression amount information of the GPNMB gene; and
step three: analyzing a change of the expression amount of the GPNMB gene to track the postoperative recurrence status of the patient with lung adenocarcinoma.

The sample contains an mRNA or a protein of the GPNMB gene and the source of the sample is not limited, for example, the sample may be derived from an adrenal gland, an appendix, a brain, a large intestine, an endometrium, an esophagus, fat, a gallbladder, a heart, kidneys, a liver, lungs, a lymph node, an ovary, a prostate, skin, a small intestine, a spleen, a stomach, a thyroid, a bladder, pleural effusion, blood, etc. However, in order to track the postoperative recurrence status of the patient with lung adenocarcinoma in real time for early treatment, the sample is preferably blood.

The amount of the sample required by the biochip of the present invention is not limited and can be adjusted and selected appropriately according to the type of the sample. In one embodiment, the amount of sample required for the biochip is a drop of blood.

The method for analyzing a change of an expression amount of a GPNMB gene of the present invention is not limited and can be adjusted according to a physiological status (age, sex, etc.) and medical history of a patient with lung adenocarcinoma. In one embodiment, a database may be established according to the physiological status of the patient with lung adenocarcinoma, a threshold value may be calculated from the database. Then the biochip of the present invention is used to measure an expression amount value of the GPNMB gene from the sample of the patient with lung adenocarcinoma. The value is compared with the threshold value. Besides, whether the sample is positive or not is determined according to a degree that the expression amount of the GPNMB gene in the sample exceeds the threshold value, for example, the sample is determined to be positive if the expression amount of the GPNMB gene exceeds 2 times of the threshold value. However, the present invention is not limited thereto.

The biochip of the present invention may track the postoperative recurrence status of all patients with lung adenocarcinoma, that is, any recurrence of lung adenocarcinoma due to drug resistance to lung adenocarcinoma-target drugs can be applicable. In one embodiment, the present invention is suitable for the lung adenocarcinoma due to a drug resistance generated by a lung adenocarcinoma-target drug of Iressa (Gefitinib), Tarceva (Erlotinib), Gilotif (Afatinib), Tagriso (Qsimetinib), Crizotinib, Certinib, Alectinib, Avastin, Cyramza, Lapatinib, etc. However, the present invention is not limited thereto.

The inventor of the present invention finds that an expression amount of the GPNMB gene in a sample of a patient with lung adenocarcinoma is relatively high, such that the biochip may track the postoperative recurrence status of all patients with lung adenocarcinoma. The inventor further finds that the expression amount of the GPNMB gene in a sample of a patient with lung adenocarcinoma resistant to a target drug, Iressa is particularly high, such that the present invention is particularly used for tracking a recurrence of a patient with lung adenocarcinoma after drug resistance to the target drug, Iressa is produced.

The relationship between the GPNMB protein and lung adenocarcinoma is described in more detail below.

The inventor of the present invention firstly finds that there may be a relationship between the GPNMB protein and lung adenocarcinoma in a cell model simulating lung adenocarcinoma, for example, in non-small cell lung cancer cells (NSCLC cells) with EGFR (L858R and L858R+T790M) mutations, the expression amount of the GPNMB at a gene level and the expression amount of the GPNMB at a protein level are both significantly higher than those of wild-type cells (WT cells).

Then in a cell experiment simulating a drug resistance produced by culturing by a target drug, Iressa, the inventor of the present invention finds that the expression amount of the GPNMB at a gene level and the expression amount of the GPNMB at a protein level also are both significantly higher than those of wild-type cells and a lung cancer cell line without drug resistance.

Besides, in an animal experiment simulating lung adenocarcinoma, the inventor finds that the expression amount of the GPNMB at a gene level and the expression amount of the GPNMB at a protein level also are both significantly higher than those of a wild-type mouse from a lung section of a mouse mutated by L858R of EGFR. The cell experiment and an animal experiment of various evidences indicate that there may be a relationship between the GPNMB protein and lung adenocarcinoma. Therefore, the inventor of the present invention further investigates whether the relationship exists in humans.

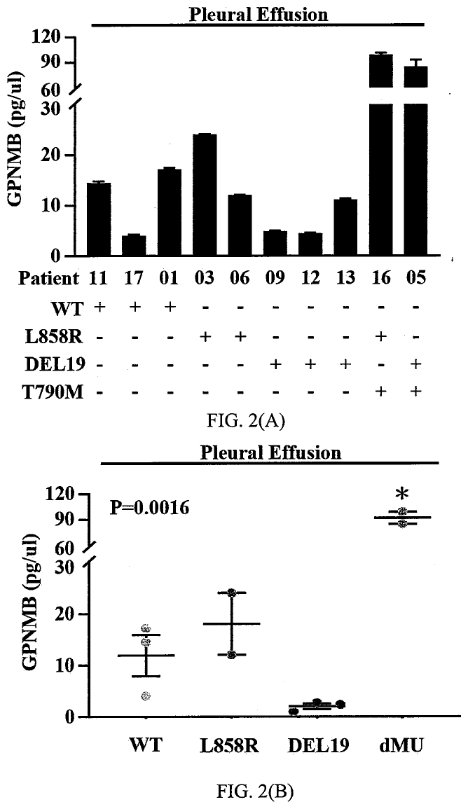

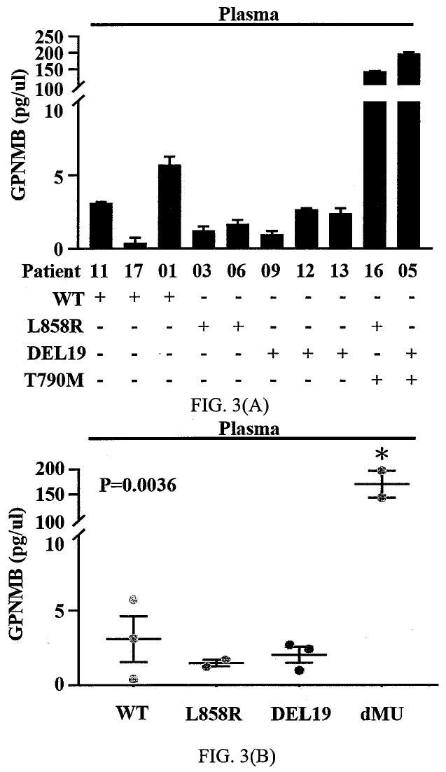

FIG. 2(A) and FIG. 2(B) show that an expression amount of a GPNMB gene is highly correlated with a patient with lung adenocarcinoma in a pleural effusion sample, and FIG. 3(A) and FIG. 3(B) show that the expression amount of the GPNMB gene is highly correlated with the patient with lung adenocarcinoma in a plasma sample. FIG. 2(A), FIG. 2(B), FIG. 3(A) and FIG. 3(B) are results of clinical tests in humans. Human patients with lung adenocarcinoma are classified. The patients containing an L858R mutation are numbered 03, 06, and 16; the patients containing a Del19 mutation are numbered 09, 12, 13, and 05; the patients containing a T790M mutation are numbered 16 and 05; and the patients not containing the above mutations are numbered 11, 17, and 01. Patient 16 who containing L858R+T790M mutations at the same time and patient 05 who containing Del19+T790M mutations at the same time produce drug resistance to the target drug, Iressa. From results of pleural effusion (FIG. 2(A) and FIG. 2(B)) and plasma (FIG. 3(A) and FIG. 3(B)) of human samples, it is known that the expression amount of the GPNMB gene is highly correlated with lung adenocarcinoma, particularly highly correlated with lung adenocarcinoma patients with an L858R mutation and L858R+T790M mutations of EGFR.

The biochip of the present invention is described by listing several experimental examples, which are not intended to limit the present invention. A person skilled in the art can make various changes and modifications without departing from the spirit and scope of the present invention. In addition, all the samples of the present invention are from National Taiwan University and Chang Gung Memorial Hospital, and other used materials are all commercially available.

### Experiment 1: Manufacture and Analysis of a GPNMB Biochip

The biochip of the present invention was prepared by using a yellow-light photolithography process (photoresist coating/exposure/development) to combine with a biomolecule coating process (molecular self-assembly/antibody (anti-GPNMB) modification) to manufacture a silicon-based working electrode which was embedded in a test paper printed by a screen and provided with a reference electrode (Ag/AgCl) and a pair of electrodes (carbon) to manufacture an electrochemical detection chip for analyzing an changing amount of the GPNMB protein.

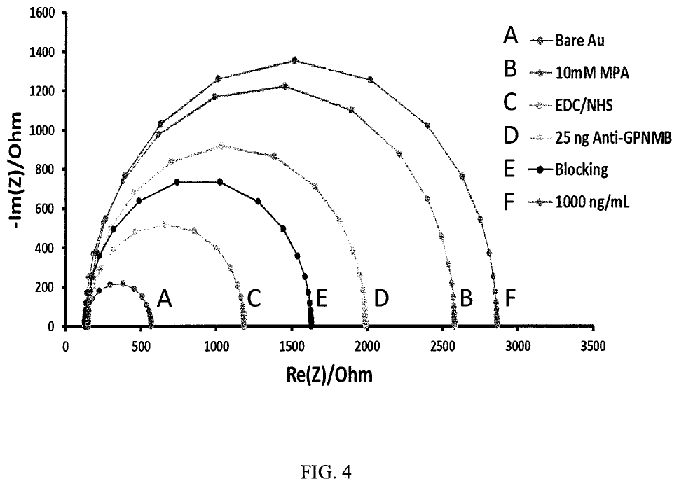

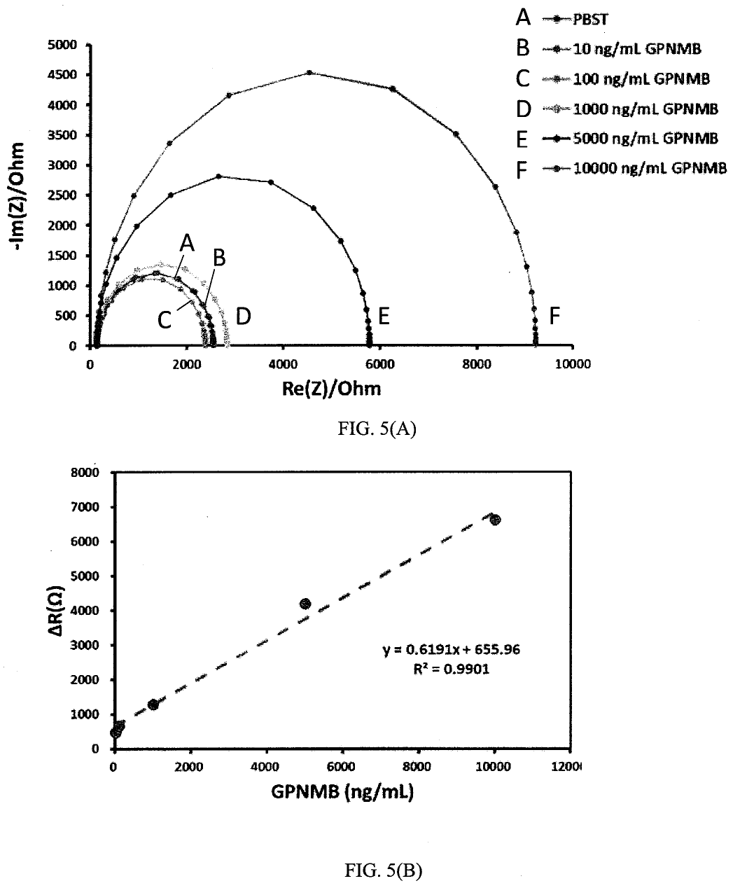

The present invention utilized a GPNMB antibody modified on a surface of a sensing electrode to capture a change amount of the GPNMB protein. Therefore, it is a primary task to determine whether the antibody is modified on the surface of the sensing electrode. Since the biochip of the present invention performs a target detection by fixing antibody modification on a biochip surface, a change of a resistance value at each modification stage is used to determine whether the modification is completed. Referring to FIG. 4, is a modification change of a resistance value at each stage according to the biomolecule coating process. As shown in the figure, a surface of a cleaned Bare Au electrode was firstly fixed with 10 mM of MPA as a linker in a molecular vapor deposition (MVD) manner, then an EDC/NHS activated functional group was used to promote linking of the antibody and the linker and fix the antibody on the biochip surface, then a blocking buffer was used to fill a gap to avoid a non-specific bonding, and finally a target antigen (GPNMB) with different concentrations, for example, the target antigen (GPNMB) of 1,000 ng/mL was used to verify the antibody modification. The verification results are shown in FIG. 5(A) and FIG. 5(B). FIG. 5(A) and FIG. 5(B) show that a resistance value change of the biochip of one embodiment of the present invention increases with an increase of an expression amount of the GPNMB protein.

Referring to FIG. 4 again, it can be seen from FIG. 4 that the resistance value of the electrode surface increases through a reaction of each stage, wherein an electron transfer is not easy due to extremely high negative electricity produced on an electrode surface after the MVD modification, the electron transfer is promoted by concentrating the positively charged antibody on the chip surface with the EDC/NHS activated functional group, thus it can be seen that the resistance value is decreased, the resistance value is improved with a subsequent increased thickness of an antigen-grafted surface, a capacitance effect is decreased, and the resistance is increased. In summary, the experiment has verified that the manufacturing method of the present invention may indeed modify the antibody on the sensing electrode, and verify that the antibody has activity and may be effectively attached to the antigen (GPNMB).

Referring to FIG. 5(A) and FIG. 5(B) again, FIG. 5(A) and FIG. 5(B) show a linear correlation between the biochip of the present invention and the content of a GPNMB protein. After 50 ng/ml of a GPNMB antibody was modified on the sensing electrode, 100, 1,000, 5,000, and 10,000 ng/ml of human GPNMB recombinant proteins (rhGPNMB) were respectively used to perform a linear verification. A resistance value change of the rhGPNMB at different concentrations in the biochip can be known from the figure (FIG. 5(A)). In addition, the present invention calculated the resistance value change (ΔR) after the rhGPNMB attachment according to the following equation (I),

$$Δ R = {R}_{A b} - {R}_{A g} \qquad [ equation ( I )$$

In equation (I), R_{Ab} is an antibody resistance value and R_{Ag} is an antigen resistance value. It can be seen from FIG. 5(B) that as the GPNMB concentration increases, the resistance change increases with a linearity of 0.99.

Then a limit of detection (LOD) of the biochip of the present invention was calculated from the following equation (II). The LOD was calculated to be 47.48 ng/ml.

$$L O D = 3 \times S D / slope \qquad [ equation ( II )$$

In equation (II), SD represents standard deviation and slope represents slope factor.

### Experiment 2: Traditional ELISA Detection Method

100 ml of a primary antibody of GPNMB was added into a 96-well plate and reacted overnight in a 4° ° C. refrigerator. After the liquid was removed, washed with 300 ml of PBS buffer for three times, remove the liquid and tapped to removing any remaining liquid, and then added 1% BSA buffer at a room temperature for one hour. After washing was performed by the PBS buffer for three times, took 100 ml of the reaction liquid and added it to the serially diluted rhGPNMB protein and specimen respectively. The mixture was sealed with a sealing film and reacted at the room temperature for two hours. Washing was performed with the PBS buffer for three times, a secondary antibody of GPNMB was added for reaction at the room temperature for two hours, and again washed with the PBS buffer for three times, added 100 ml of 200-fold diluted HRP buffer, incubated the mixture for 30 minutes at the room temperature in the dark, washing was performed with the PBS buffer for three times, added 100 ml of a substrate solution and incubated at the room temperature for 30 minutes in the dark, and finally, after 50 μl of a stop solution was added, the materials were uniformly mixed, a result was analyzed by using an ELISA reader at a wavelength of 450 nm.

### Experiment 3: Detection Change of GPNMB Biochip in Cell Sample

The experiment used lung adenocarcinoma cell line samples with relatively uncomplicated sample components, namely wild-type (WT) cells, EGFR mutation (EGFR MU) cells and cells with drug resistance to the target drug, Iressa (L858R+T790M or Del19+T790M, hereinafter abbreviated as EGFR dMU) for analysis. The cells were tested by using the biochip of the present invention. It was found that the biochip of the present invention may distinguish differences between WT and EGFR MU and EGFR dMU, such that pleural effusion of patients with lung adenocarcinoma was further used for testing.

### Experiment 4: Detection Change of GPNMB Biochip in Pleural Effusion Sample

The present embodiment used samples of human patients for an experiment. Information of each patient was shown in Table 1 below. Referring to FIG. 2 again, the experiment collected pleural effusion samples from 10 patients with lung adenocarcinoma (No. 1, No. 3, No. 5, No. 6, No. 9, No. 11, No. 13, No. 15, No. 16, and No. 17) and analyzed the expression amount of the GPNMB protein by the conventional ELISA. It can be seen from the figure that these patients with lung adenocarcinoma respectively contained WT, EGFR MU, and EGFR dMU, wherein GPNMB expression amounts of patients with EGFR dMU (No. 05 and No. 16) were much higher than those of patients with WT and EGFR MU. From the above experiment, it shows that when the patients with lung adenocarcinoma produced resistance to the target drug, Iressa, the expression amount of the GPNMB protein in pleural effusion would increase greatly.

TABLE 1
Cancer type and EGFR status of patients

| Patient No. | Cancer | Cancer cell type | EGFR status |
|---|---|---|---|
| 01 | + | Adenosquamous | WT |
| 02 | − | − | − |
| 03 | + | Adeno | L858R |
| 04 | + | Squamous | WT |
| 05 | + | Adeno | 19DEL/T790M |
| 06 | + | Adeno | L858R |
| 07 | + | Small cell | − |
| 08 | + | Plasmacytoid dendritic cells | − |
| 09 | + | Adeno | 19DEL |
| 11 | + | Adeno | WT |
| 12 | + | Adeno | 19DEL |
| 14 | + | Squamous | − |
| 15 | + | Adeno | 19DEL |
| 13 | + | Adeno | 19DEL |
| 16 | + | Adeno | L858R/T790M |
| 17 | + | Adeno | WT |

+ indicates that a cancer test is positive and − indicates that a cancer test is negative.

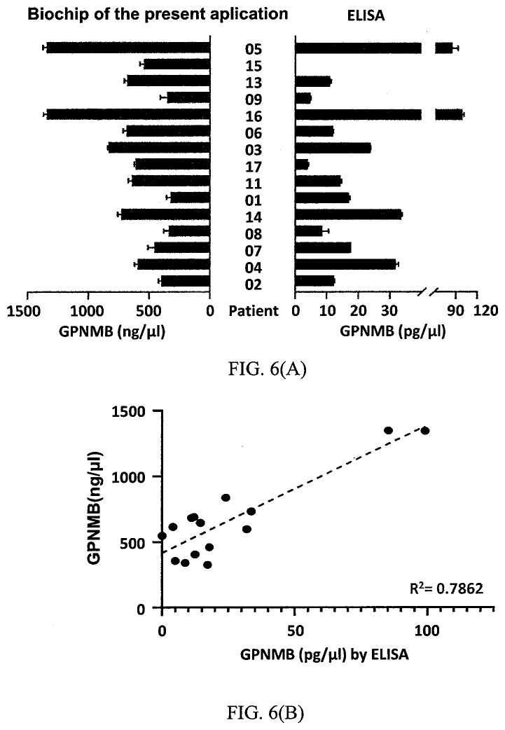

Then 15 samples (samples of patients except patient No. 12 in Table 1) were collected, wherein 10 samples were patients with lung adenocarcinoma. These pleural effusion samples were analyzed by the biochip of the present invention and compared with the conventional ELISA method. The results were shown in FIG. 6(A). The histogram respectively presented the concentration of the GPNMB protein in the samples of the patients with the lung adenocarcinoma, which was then compared with the results measured by the conventional ELISA. It was found that a whole changing trend of the detection results of the biochip of the present invention was similar to the results of the conventional ELISA method. Referring to FIG. 6(B), the correlation between the biochip of the present invention and the conventional ELISA method, R²=0.78. The expression amounts of EGFR dMU (patient No. 05 and 16) were significantly different from those of other groups. The above experiment showed that the detection accuracy of the biochip of the present invention was similar to the result of the conventional ELISA method, and especially the biochip may be used for analyzing a drug-resistant patient with the lung adenocarcinoma. FIG. 6(A) and FIG. 6(B) show comparisons between the biochip according to one embodiment of the present invention and a conventional ELISA method.

### Experiment 5: Detection Change of GPNMB Biochip in Plasma Sample

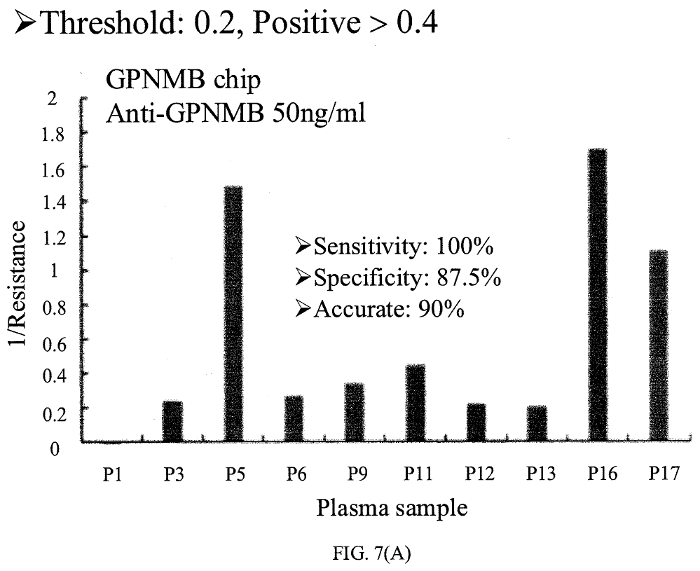

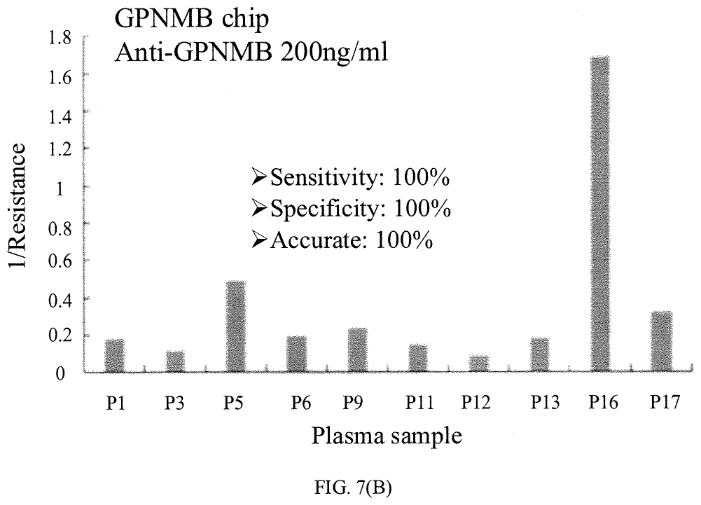

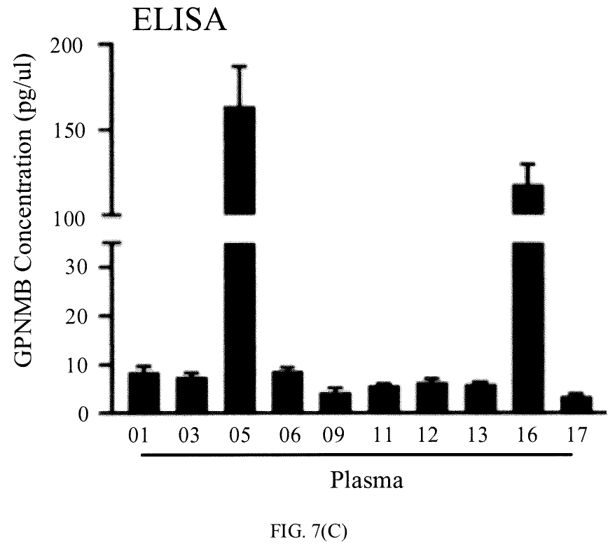

The present invention further tested 10 plasma samples of patients with lung adenocarcinoma (No. 1, No. 3, No. 5, No. 6, No. 9, No. 11, No. 13, No. 15, No. 16, and No. 17) and adjusted a concentration change of an antibody to obtain an optimized detection result. Following the previous experiment, the biochip of the present invention analyzed a result by measuring a reciprocal value of a resistance value and a threshold value (1/resistance) was further divided. The results were shown in FIG. 7(A), FIG. 7(B) and FIG. 7(C). It can be seen from FIG. 7(A), the detection results of plasma samples from patients with lung adenocarcinoma by different antibody concentrations were analyzed. When the threshold value was set to 0.2, the detection result of 2 times or more of the threshold value was considered to be positive. When the antibody concentration on the biochip was 50 ng/ml, the sensitivity of the biochip was 100%, the specificity was 87.5%, and the accuracy was 90%. As can be seen from FIG. 7(B), when the antibody concentration on the biochip was 200 ng/ml, the sensitivity, specificity and accuracy of the biochip were all 100%. Referring to FIG. 7(C), it was demonstrated that a performance of the detection of the biochip of the present invention was consistent with the detection result of the conventional ELISA method. It was demonstrated that the chip may be used for tracking drug resistance of blood samples in lung adenocarcinoma.

The above results were combined. The biochip of the present invention for tracking the postoperative recurrence status of a patient with lung adenocarcinoma may be used for tracking of the postoperative recurrence of the patient with lung adenocarcinoma, particularly used for tracking the postoperative recurrence after development of drug resistance to a target drug, Iressa. The sensitivity, specificity and accuracy of the biochip were all 100%. Compared with the traditional ELISA test method, the biochip of the present invention can achieve an effect of a quick examination by using a sample of one drop of blood, track the postoperative recurrence status of a patient with lung adenocarcinoma in real time so as to perform early treatment, and reduces a medical cost of the patient with lung adenocarcinoma.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A biochip for tracking postoperative recurrence status of a patient with lung adenocarcinoma, wherein the biochip comprises a bare plate layer, the bare plate layer comprises a sensing electrode, and the sensing electrode comprises a biological agent capable of measuring an expression amount of a GPNMB gene.

2. The biochip for tracking the postoperative recurrence status of a patient with lung adenocarcinoma according to claim 1, wherein the biological agent measures an expression amount of mRNA or protein of the GPNMB gene.

3. The biochip for tracking the postoperative recurrence status of a patient with lung adenocarcinoma according to claim 1, wherein the biological agent is an antibody specifically binding to a protein of the GPNMB gene.

4. The biochip for tracking the postoperative recurrence status of a patient with lung adenocarcinoma according to claim 3, wherein the antibody has a concentration of 20 ng/ml-5 μg/ml.

5. The biochip for tracking the postoperative recurrence status of a patient with lung adenocarcinoma according to claim 1, wherein a limit of detection is 0.5 ng/ml-70 ng/ml.

6. The biochip for tracking the postoperative recurrence status of a patient with lung adenocarcinoma according to claim 1, wherein the biochip further comprises a lower cover layer, a middle interlayer, and an upper cover layer, the lower cover layer is arranged at a lower side of the bare plate layer, and the middle interlayer and the upper cover layer are arranged on an upper side of the bare plate layer.

7. The biochip for tracking the postoperative recurrence status of a patient with lung adenocarcinoma according to claim 6, wherein the bare plate layer comprises a plurality of electric wires, a relative electrode and a reference electrode, the middle interlayer comprises a micro-channel, and the upper cover layer comprises a sample dripping area; and the micro-channel is arranged correspondingly to a position of the sample dripping area, such that a sample can flow from the sample dripping area to the sensing electrode.

8. A method for tracking the postoperative recurrence status of a patient with lung adenocarcinoma, comprising the following steps: step one, providing a sample from a patient with lung adenocarcinoma; step two: contacting the sample with a carrier capable of detecting an expression amount of a GPNMB gene to obtain expression amount information of the GPNMB gene; and step three: analyzing a change of the expression amount of the GPNMB gene to track the postoperative recurrence status of the patient with lung adenocarcinoma.

9. The method according to claim 8, wherein the sample is blood; and the expression amount of the GPNMB gene is a protein expression amount of the GPNMB gene.

10. The method according to claim 8, wherein the patient with lung adenocarcinoma has a mutation of L858R or L858R+T790M of EGFR; and the postoperative recurrence is due to development of drug resistance of the patient with lung adenocarcinoma to a target drug, Iressa.
